(19) United States
(12) Patent Application Publication
(10) Pub. No.: US 2018/0111106 A1
(43) Pub. Date: Apr. 26, 2018

(54) Method for Producing Microparticles

(71) Applicant: METAL INDUSTRIES RESEARCH & DEVELOPMENT CENTRE, Kaohsiung City (TW)

(72) Inventors: Zong-Hsin Liu, Kaohsiung City (TW); Cheng-Han Hung, Kaohsiung City (TW); Ying-Chieh Lin, Kaohsiung City (TW); Cheng-Tang Pan, Kaohsiung City (TW); Yao-Kun Huang, Kaohsiung City (TW); Ying-Cheng Lu, Kaohsiung City (TW)

(21) Appl. No.: 15/379,764

(22) Filed: Dec. 15, 2016

(30) Foreign Application Priority Data
Oct. 26, 2016 (TW) 105134609

(51) Int. Cl. B01J 13/06; A61K 9/16

(52) U.S. Cl. CPC B01J 13/06; A61K 9/1682

(57) ABSTRACT

A method for producing microparticles includes filling a tank with a first fluid. A nozzle including a plurality of first outlet ports facing the tank is provided. A second fluid forms a plurality of liquid films on the first outlet ports. The liquid films on the first outlet ports absorb a vibrational energy to form a plurality of microdroplets that falls into the first fluid. The first fluid envelops outer layers of the microdroplets to form a plurality of semi-products of microparticles. Each semi-product includes an outer layer formed by the first fluid and an inner layer formed by the second fluid. The semi-products in the tank are collected. The outer layers of the semi-products are removed to form a plurality of microparticle products.

### CROSS REFERENCE TO RELATED APPLICATIONS

The application claims the benefit of Taiwan application serial No. 105134609, filed Oct. 26, 2016, the subject matter of which is incorporated herein by reference.

### BACKGROUND

### 1. Technical Field

The present disclosure relates to a method for producing microparticles and, more particularly, to a method for mass production of microparticles.

### 2. Description of the Related Art

Microparticles, also known as microspheres, are spherical particles having a diameter ranging from 1 μm to 1000 μm, are generally used as microcarriers for releasing drug, and have become one of the emerging drug delivery technologies due to the characteristics of targeting, controlled release, stability, and surface modifiability.

Since the diameters of microparticles are small, the first aim is to form microparticles of uniform diameters to make each microparticle have the same drug releasing effect. For example, a conventional micro fluid passageway structure 9 shown in FIG. 1 can be used to form microparticles with more uniform diameters.

With reference to FIG. 1, the conventional micro fluid passageway structure 9 includes a Y-shaped passageway 91, a curing agent filling port 92, a material solution filling port 93, and a cruciform micro fluid passageway 94. The Y-shaped passageway 91 is intercommunicated with the cruciform micro fluid passageway 94. A branch of the Y-shaped passageway 91 is intercommunicated with the curing agent filling port 92 through which a curing agent solution is filled. Another branch of the Y-shaped passageway 91 is intercommunicated with the material solution filling port 93 through which a material solution is filled. The curing agent solution and the material solution form a pre-solidified mixed solution at a third end of the Y-shaped passageway 91. The third end of the Y-shaped passageway 91 is intercommunicated with the cruciform micro fluid passageway 94. A water phase solution is filled through two ends of the cruciform micro fluid passageway 94. The shear stress of the water phase solution filled into the cruciform micro fluid passageway 94 makes the pre-solidified mixed solution flowing into the cruciform micro fluid passageway 94 form emulsified spheres separate from each other, and each emulsified sphere finally forms a microparticle.

Although the above conventional micro fluid passageway structure 9 can form microparticles with more uniform diameters, the conventional micro fluid passageway structure 9 cannot easily proceed with mass production. Improvement is, thus, necessary.

### SUMMARY

To solve the above problem, the present disclosure provides a method for producing microparticles to enable mass production of microparticles.

A method for producing microparticles according to the present disclosure includes filling a tank with a first fluid; providing a nozzle including a plurality of first outlet ports facing the tank; making a second fluid form a plurality of liquid films on the plurality of first outlet ports; making each of the plurality of liquid films on the plurality of first outlet ports absorb a vibrational energy, forming a plurality of microdroplets that falls into the first fluid; making the first fluid envelop outer layers of the plurality of microdroplets to form a plurality of semi-products of microparticles, with each of the plurality of semi-products of microparticles including an outer layer formed by the first fluid and an inner layer formed by the second fluid; and collecting the plurality of semi-products of microparticles in the tank and removing the outer layers of the plurality of semi-products of microparticles to form a plurality of microparticle products. Thus, the method for producing microparticles according to the present disclosure directionally sprays microdroplets of a uniform size out of the outlet ports, and the microdroplets fall into the tank. Thus, the present disclosure achieves the effect of mass production of microparticles of a uniform size.

In an example, each of the plurality of liquid films formed by the second fluid on the plurality of first outlet ports is a single-layer liquid film. Each of the plurality of microdroplets is a single-layer microdroplet formed by one of the single-layer liquid films. The single-layer microdroplets fall into the first fluid. Each of the plurality of semi-products of microparticles is comprised by the outer layer and the inner layer. Each of the plurality of microparticle products includes only the inner layer formed by the second fluid. Thus, mass production of single-layer microparticles of a uniform size is permitted.

In another example, the second fluid and a third fluid together form a plurality of dual-layer liquid films on the plurality of first outlet ports. The plurality of dual-layer liquid films forms a plurality of dual-layer microdroplets that falls into the first fluid. Each of the plurality of semi-products of microparticles further includes a central layer formed by the third fluid. The inner layer is located between the outer layer and the central layer. Each of the plurality of microparticle products includes a shell layer formed by the second fluid and a core layer formed by the third fluid. In an example, the nozzle includes a tube assembly. The tube assembly includes a first tube and a second tube surrounded by the first tube. A first fluid passageway is defined between the first tube and the second tube. A second fluid passageway is defined in the second tube. The first tube includes a first end forming a first filling port intercommunicated with the first fluid passageway and a second end forming the plurality of first outlet ports intercommunicated with the first fluid passageway. The second tube includes a first end forming a second filling port and a second end forming a second outlet port. A formation space is defined between the second outlet port and the plurality of first outlet ports. The third fluid forms a single-layer liquid film in the second outlet port. The second fluid envelops and shears the single-layer liquid film formed in the second outlet port, thereby forming the plurality of dual-layer liquid films on the plurality of first outlet ports. The second fluid flows in the first fluid passageway toward the plurality of first outlet ports at a first speed. The third fluid flows through the second fluid passageway toward the second outlet port at a second speed. The first speed is greater than the second speed. Thus, mass production of dual-layer microparticles of a uniform size is permitted.

In an example, the nozzle includes a piezoelectric portion and an amplifying portion connected to the piezoelectric portion. High frequency electric energy generated by a supersonic wave generator is transmitted to the piezoelectric portion and is converted by the piezoelectric portion into vibrational energy. The amplifying portion makes the plurality of liquid films on the plurality of first outlet ports absorb the vibrational energy. Thus, mass production of microparticles of a uniform size is permitted.

In an example, the nozzle includes a nozzle body having a first end and a second end opposite to the first end. The second fluid flows from the first end toward the second end of the nozzle body and forms the plurality of liquid films on the plurality of first outlet ports.

In an example, the second fluid is a biodegradable polymer, and the third fluid is a fluid mixed with an active pharmaceutical ingredient in a particle or powder form. Alternatively, the second fluid is a biodegradable polymer, and the third fluid is a fluid mixed with an active pharmaceutical ingredient in a liquid form. Thus, when the microparticle products are given to an organism, a slow releasing effect of the active pharmaceutical ingredient is achieved by enveloping of the biodegradable polymer.

The present disclosure will become clearer in light of the following detailed description of illustrative embodiments of the present disclosure described in connection with the drawings.

### BRIEF DESCRIPTION OF THE DRAWINGS

FIG. 1 is a diagrammatic view of a conventional micro fluid passageway structure.

FIG. 2 is a diagrammatic view illustrating a method for producing microparticles of a first embodiment according to the present disclosure.

FIG. 3 is a diagrammatic view of an example of a semi-product of a microparticle produced by the method illustrated in FIG. 2.

FIG. 4 is a diagrammatic view of a microparticle product of FIG. 3.

FIG. 5 is a diagrammatic view of another example of a semi-product of a microparticle produced by the method illustrated in FIG. 2.

FIG. 6 is a diagrammatic view of a microparticle product of FIG. 5.

FIG. 7 is a diagrammatic view illustrating a method for producing microparticles of a second embodiment according to the present disclosure.

FIG. 8 is a diagrammatic view of an example of a semi-product of a microparticle produced by the method illustrated in FIG. 7.

FIG. 9 is a diagrammatic view of a microparticle product of FIG. 8.

FIG. 10 is a diagrammatic view of another example of a microparticle product produced by the method illustrated in FIG. 7.

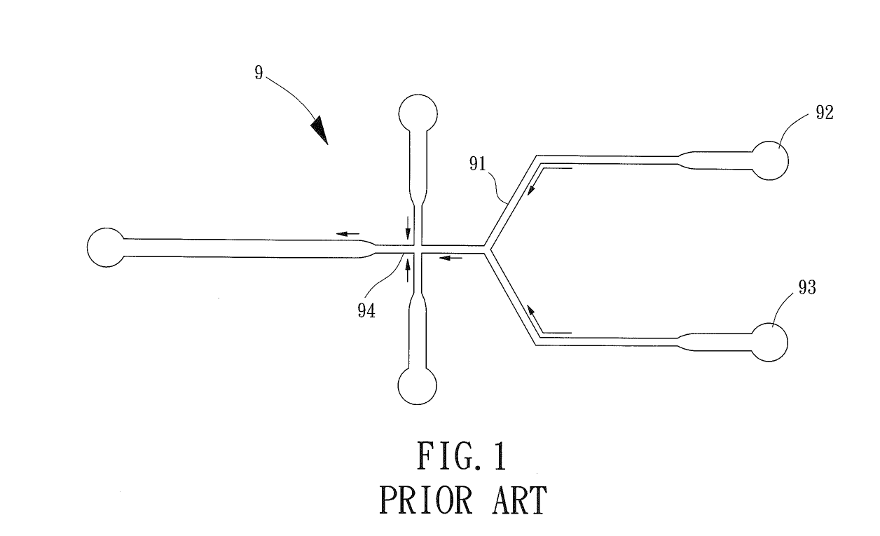

### DETAILED DESCRIPTION

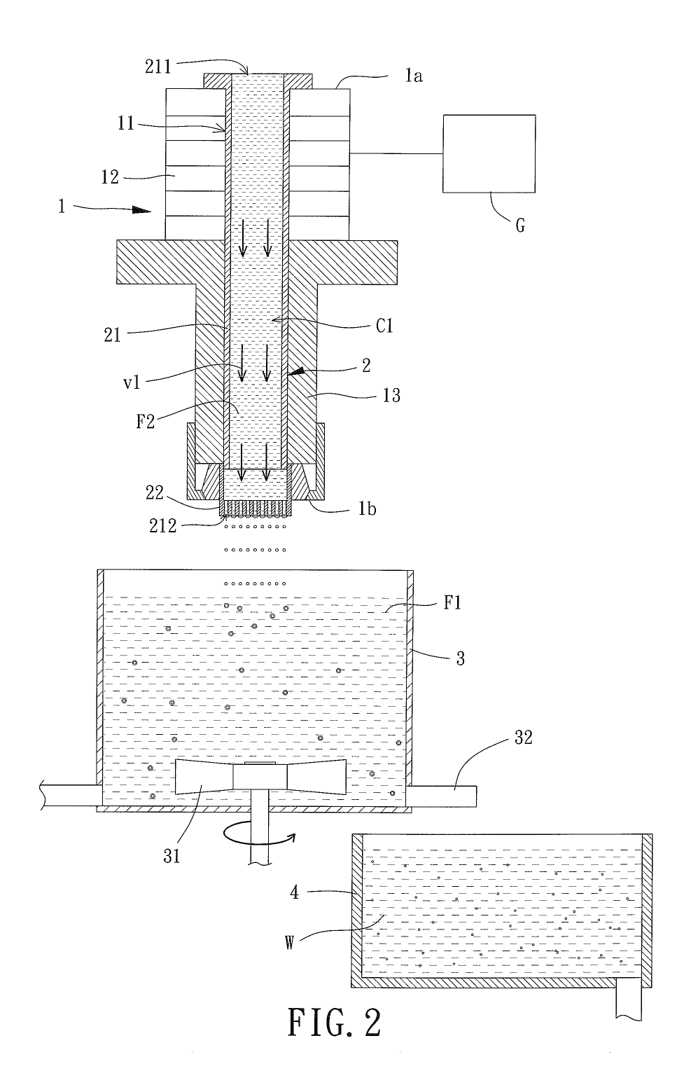

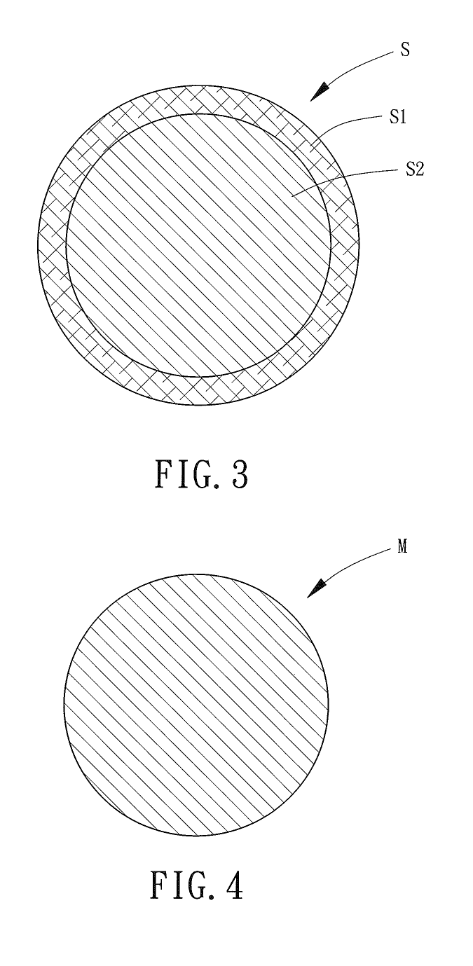

With reference to FIG. 2, a method for producing microparticles according to the present disclosure makes a plurality of microdroplets fall into a first fluid F1, makes the first fluid F1 envelop an outer layer of each microdroplet (namely, emulsification) to form a semi-product S of a microparticle (see FIG. 3) having an outer layer S1 formed by the first fluid F1, and removes the outer layer S1 of each semi-product S to form a microparticle product M.

Still referring to FIG. 2, specifically, the present disclosure uses a nozzle to accomplish the above-mentioned method for producing microparticles. The nozzle can form the microdroplets that fall into the first fluid F1.

The nozzle includes a nozzle body 1 and a tube assembly 2. The nozzle body 1 includes a through-hole 11. The tube assembly 2 is mounted in the through-hole 11.

The nozzle body 1 has a first end 1a and a second end 1b opposite to the first end 1a. The nozzle body 1 further includes an oscillating device and an amplifying portion 13. The oscillating device can be directly or indirectly connected to the amplifying portion 13. The amplifying portion 13 is located between the first end 1a and the second end 1b. The through-hole 11 extends from the first end 1a through the amplifying portion 13 and extends through the second end 1b. In this embodiment, the oscillating device includes a piezoelectric portion 12. When the piezoelectric portion 12 receives high frequency electric energy from a supersonic wave generator the high frequency electric energy is turned into vibrational energy which is transmitted to the amplifying portion 13, such that the second end 1b of the nozzle body 1 can have the maximum vibrational amplitude. In this embodiment, the piezoelectric portion 12 is directly connected to the amplifying portion 13, and the through-hole 11 extends from the first end 1a through the piezoelectric portion 12 and the amplifying portion 13 in sequence and extends through the second end 1b. Thus, the contact area between the piezoelectric portion 12 and the amplifying portion 13 can be increased to effectively transmit the vibrational energy to the amplifying portion 13.

The tube assembly 2 includes an interior forming a first fluid passageway C1. In this embodiment, the tube assembly 2 includes a first tube 21 in which the first fluid passageway C1 is defined to permit a second fluid F2 to flow from the first end 1a toward the second end 1b of the nozzle body 1.

The first tube 21 can be formed by a material capable of resisting adhesion of the second fluid F2. Alternatively, a coating capable of resisting adhesion of the second fluid F2 can be coated on an inner periphery of the first tube 21 to increase flow smoothness of the second fluid F2 in the first fluid passageway C11. Furthermore, the flow rate and pressure of the second fluid F2 must be considered when determining the diameter of the first tube 21. Furthermore, the pressure change of the second fluid F2 is more sensitive when the diameter of the first tube 21 is smaller, providing a better micro flow control effect.

Furthermore, a first filling port 211 is defined in a first end of the first tube 21, and a plurality of first outlet ports 212 is defined in a second end of the first tube 21. The first filling port 211 and the first outlet ports 212 are intercommunicated with the first fluid passageway C1. In this embodiment, an end of the first tube 21 is formed by a sleeve 22 including the first outlet ports 212. Thus, a worker can replace the tube assembly 2 or the sleeve 22 according to different needs to improve use convenience. Furthermore, it is not necessary to replace the whole nozzle, thereby reducing the purchasing costs of the nozzle.

Thus, a worker can fill the second fluid F2 into the first filling port 211, such that the second fluid F2 flows through the first fluid passageway C1 at a first speed v1 and forms a liquid film on each first outlet port 212 by surface tension of the second fluid F2 (as shown in the FIG. 2, the liquid film is a single-layer liquid film). Furthermore, the single-layer liquid film formed on each first outlet port 212 can absorb the vibrational energy generated by the combined action of the piezoelectric portion 12 and the amplifying portion 13 to form a standing wave, thereby reducing the thickness of the single-layer liquid film. When the vibrational energy absorbed by the single-layer liquid film on each first outlet port 212 exceeds the surface tension of the single-layer liquid film, each liquid film can exit the corresponding first outlet port 212 in the form of uniform and tiny spray, which will be described in detail hereinafter. For the sake of explanation, the second fluid F2 exiting the first outlet ports 212 in the form of spray is hereinafter referred to as “microdroplet”.

The diameter dₚ of the microdroplet can be expressed by the equation presented by Robert J. Lang in 1962.

dₚ=0.34·λ

λ=((8·π·θ)/(ρ·f²))^{1/3}

wherein λ is the wavelength of the standing wave, θ is the surface tension of the second fluid F2, ρ is the density of the second fluid F2, and f is the vibrational frequency. As can be seen from the above equation, a smaller diameter of the microdroplet can be obtained by simply increasing the vibrational frequency.

The microdroplets can fall into the first fluid F1 received in a tank 3. Thus, the first fluid F1 envelops the outer layer of each microdroplet by emulsification to form a semi-product S (see FIG. 3) in the tank 3. Each semi-product S consists of an outer layer S1 formed by the first fluid F1 and an inner layer S2 formed by the second fluid F2. A person skilled in the art can choose the first fluid F1 and the second fluid F2 according to needs. Detailed description is not given to avoid redundancy.

Furthermore, a rotating member 31 mounted in the tank 3 can be adjustably rotated to drive the first fluid F1 to create a speed, such that the second fluid F2 in the form the microdroplets falling into the tank 3 can generate a frictional contact with the first fluid F1. Thus, the semi-product S can be sheared into a smaller size.

Next, the semi-products S in the tank 3 are collected and dried by hot air to evaporate the outer layers S1 formed by the first fluid F1, forming the products of microparticles M merely formed by the second fluid F2 (see FIG. 4). Alternatively, the semi-products S are washed by an aqueous solution W to remove the outer layers S1, forming microparticle products M merely formed by the second fluid F2. Specifically, in this embodiment, the tank 3 is connected by an outlet pipe 32 to a collection tank 4 that receives the aqueous solution W for washing the semi-products S. Thus, the first fluid F1 along with the semi-products S can flow through the outlet pipe 32 into the collection tank 4, and a worker can collect the microparticle products M in the collection tank 4.

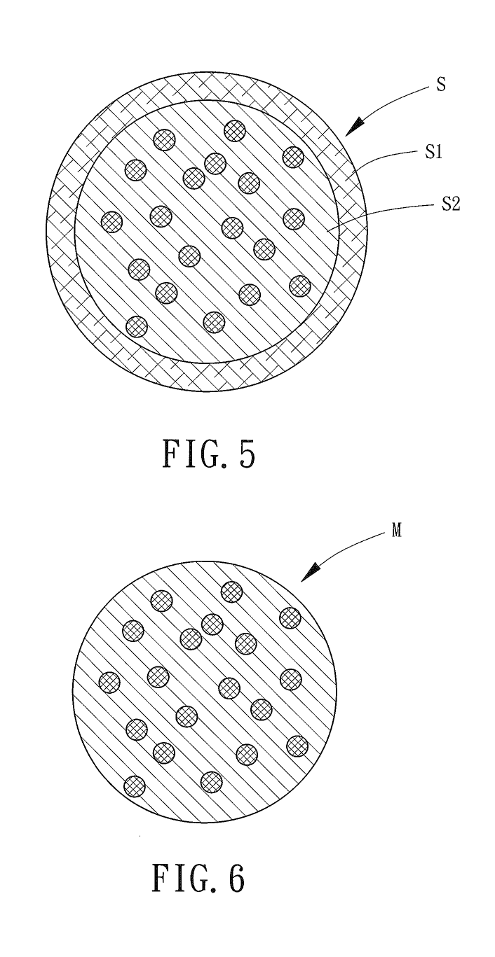

Furthermore, the worker can change the composition of the second fluid F2 to form the semi-products S (see FIG. 5) in the tank 3 and to subsequently form the microparticle products M shown in FIG. 6. Specifically, the second fluid F2 can be a biodegradable polymer mixed with an active pharmaceutical ingredient in a particle or powder form by emulsification. Thus, when the microparticle products M are given to an organism, a slow releasing effect of the active pharmaceutical ingredient is achieved by enveloping of the biodegradable polymer. For example, the biodegradable polymer can be aliphatic polyesters, aliphatic-aromatic copolyesters, polylactide-aliphatic copolyesters, polycaprolactone, polyglutamic acid, poly-hydroxy acid ester, or polylactide. Preferably, aliphatic polyesters can be polyglycolic acid, polybutylene succinate butanediamine, or polyethylene succinate. Aliphatic-aromatic copolyesters can be polyethylene terephthalate-polyoxyethylene. Polylactide-aliphatic copolyesters can be polylactic glycolic acid.

Based on the same technical concept, the method for producing microparticles according to the present disclosure can produce multi-layer microparticle products M by using the tube assembly 2 of the nozzle, which will be described in detail hereinafter.

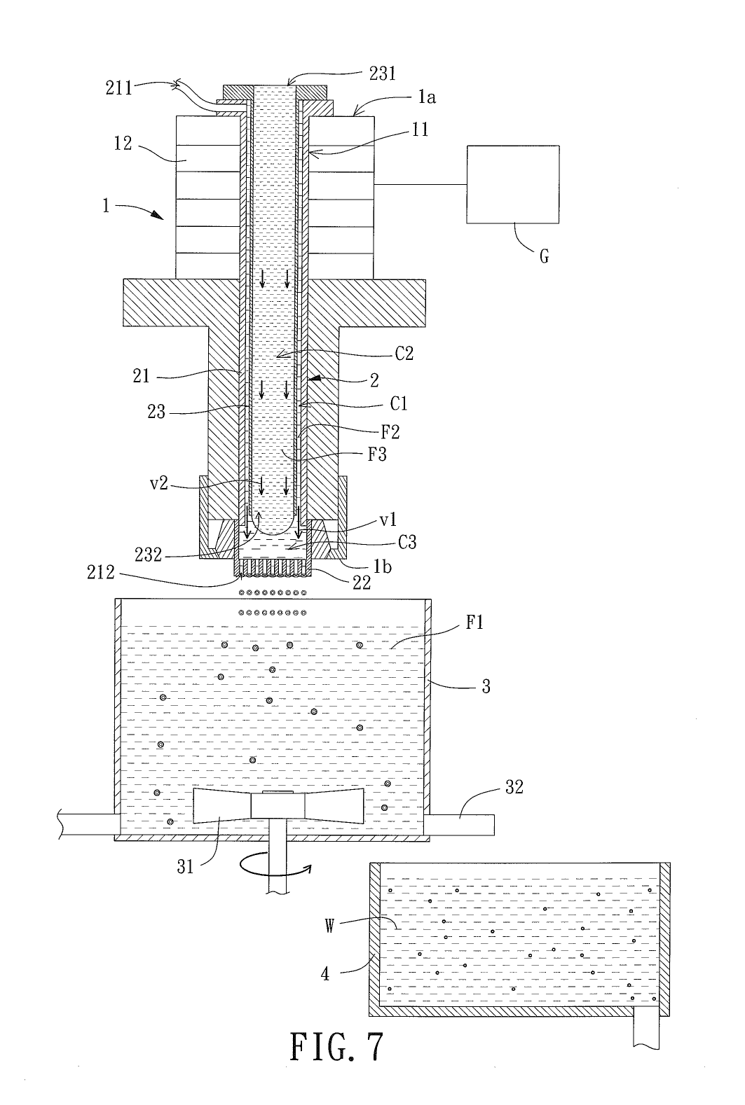

With reference to FIG. 7, the tube assembly 2 further includes a second fluid passageway C2. In an example, the tube assembly 2 further includes a second tube 23 surrounded by the first tube 21. The first fluid passageway C1 is defined between the first tube 21 and the second tube 23. The second fluid passageway C2 is defined in the second tube 23 and permits a third fluid F3 to flow from the first end 1a toward the second end 1b of the nozzle body 1.

A first end and a second end of the second tube 23 form a second filling port 231 and a second outlet port 232, respectively. The second filling port 231 and the second outlet port 232 are intercommunicated with the second fluid passageway C2. Thus, the worker can fill the third fluid F3 into the second filling port 231, and the third fluid F3 flows through the second fluid passageway C2 at a second speed v2 and forms a liquid film on the second outlet port 232 by surface tension of the third fluid F3.

It is noted that in order to make the third fluid F3 form a complete liquid film on the second outlet port 232 and make the second fluid F2 envelop the liquid film formed by the third fluid F3, a formation space C3 is preferably defined between the second outlet port 232 of the second tube 23 and the first outlet ports 213 of the first tube 21. The formation space C3 is intercommunicated with the second outlet port 232 of the second tube 23 and the first outlet ports 213 of the first tube 21.

Therefore, when the worker fills the third fluid F3 into the second fluid passageway C2 at the second speed v2 to make the third fluid F3 form a single-layer liquid film on the second outlet port 232 and fills the second fluid F2 into the first fluid passageway C1 at the first speed v1 greater than the second speed v2, a shear force is generated by the difference between the first speed v1 and the second speed v2. Thus, the second fluid F2 in the formation space C3 envelopes and shears the single-layer liquid film formed by the third fluid F3 on the second outlet port 232. Furthermore, dual-layer liquid films are formed on the first outlet ports 212 by surface tension.

Furthermore, the dual-layer liquid film formed on each first outlet port 212 absorbs the vibrational energy generated by the combined action of the piezoelectric portion 12 and the amplifying portion 13 and forms a standing wave to reduce the thickness of the liquid film. Furthermore, when the vibrational energy absorbed by the dual-layer liquid film on each first outlet port 212 exceeds the surface tension of the dual-layer liquid film, a plurality of dual-layer microdroplets of a uniform size is sprayed directionally outward from first outlet ports 212 and falls into the tank 3.

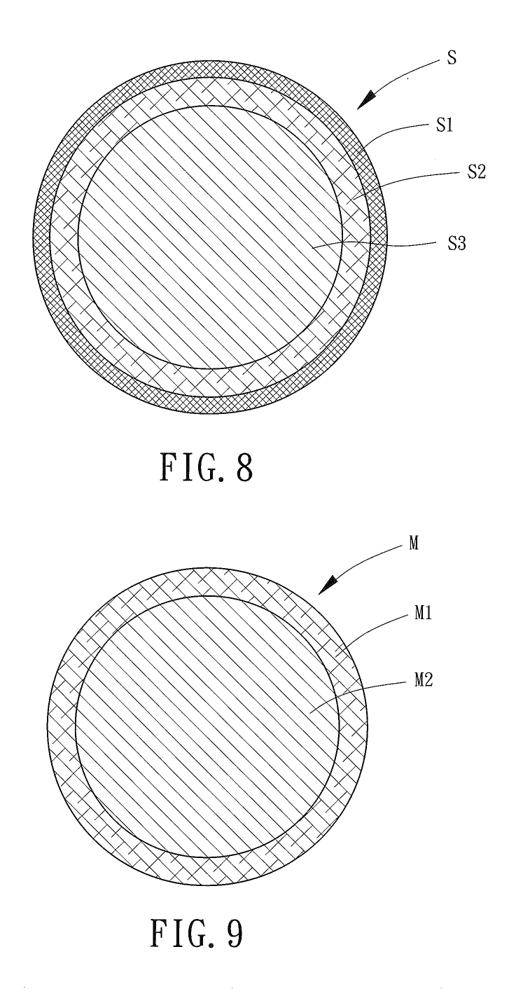

At this time, the first fluid F1 in the tank 3 envelops the outer layer of each dual-layer microdroplet by emulsification to form a semi-product S (see FIG. 8) in the tank 3. The semi-product S includes an outer layer S1 formed by the first fluid F1, an inner layer S2 formed by the second fluid F2, and a central layer S3 formed by the third fluid F3. The inner layer S2 is located between the outer layer S1 and the central layer S3. A person skilled in the art can choose the first fluid F1, the second fluid F2, and the third fluid F3 according to needs. Detailed description is not given to avoid redundancy.

Next, the semi-products S are dried by hot air to evaporate the outer layers S1 formed by the first fluid F1, forming the products of microparticles M (FIG. 9). Alternatively, the semi-products S are washed by an aqueous solution W to remove the outer layers S1, forming microparticle products M (FIG. 9). Each microparticle product M includes a shell layer M1 formed by the second fluid F2 and a core layer M2 formed by the third fluid F3.

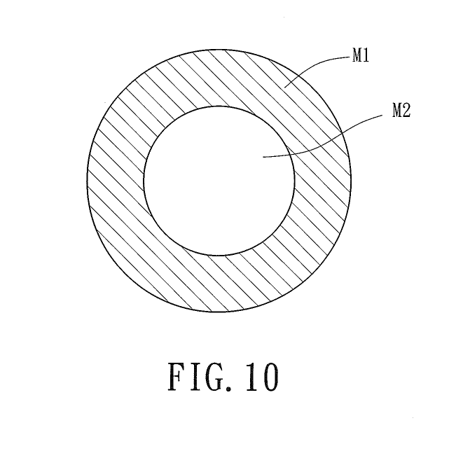

Furthermore, the worker can change the composition of the third fluid F3 to form the microparticle products M. For example, to form the microparticle product M shown in FIG. 9, the second fluid F2 can be a biodegradable polymer, the third fluid F3 can be a fluid mixed with an active pharmaceutical ingredient in a liquid form. Moreover, in a case that a gaseous fluid is used as the third fluid F3, a microparticle product M shown in FIG. 10 can be formed.

Based on the same technical concept, the worker can use a tube assembly 2 including a third tube (not shown) received in the second tube 22 to produce multi-layer microparticles having more than two layers, which can be appreciated by a person having ordinary skill in the art without redundant description.

In view of the foregoing, the method for producing microparticles according to the present disclosure directionally sprays microdroplets of a uniform size out of the outlet ports 212, and the microdroplets fall into the tank 3. Thus, the present disclosure achieves the effect of mass production of microparticles of a uniform size.

Thus since the present disclosure disclosed herein may be embodied in other specific forms without departing from the spirit or general characteristics thereof, some of which forms have been indicated, the embodiments described herein are to be considered in all respects illustrative and not restrictive. The scope of the present disclosure is to be indicated by the appended claims, rather than by the foregoing description, and all changes which come within the meaning and range of equivalency of the claims are intended to be embraced therein.

What is claimed is:

1. A method for producing microparticles, comprising:
filling a tank with a first fluid;
providing a nozzle including a plurality of first outlet ports facing the tank;
making a second fluid form a plurality of liquid films on the plurality of first outlet ports;
making each of the plurality of liquid films on the plurality of first outlet ports absorb a vibrational energy, forming a plurality of microdroplets that falls into the first fluid;
making the first fluid envelop outer layers of the plurality of microdroplets to form a plurality of semi-products of microparticles, with each of the plurality of semi-products of microparticles including an outer layer formed by the first fluid and an inner layer formed by the second fluid; and
collecting the plurality of semi-products of microparticles in the tank and removing the outer layers of the plurality of semi-products of microparticles to form a plurality of microparticle products.

2. The method for producing microparticles as claimed in claim 1, with each of the plurality of liquid films formed by the second fluid on the plurality of first outlet ports being a single-layer liquid film, with each of the plurality of microdroplets being a single-layer microdroplet formed by one of the single-layer liquid films, with the single-layer microdroplets falling into the first fluid, with each of the plurality of semi-products of microparticles consisting of the outer layer and the inner layer, and with each of the plurality of microparticle products including only the inner layer formed by the second fluid.

3. The method for producing microparticles as claimed in claim 1, with the second fluid and a third fluid together forming a plurality of dual-layer liquid films on the plurality of first outlet ports, with the plurality of dual-layer liquid films forming a plurality of dual-layer microdroplets that falls into the first fluid, with each of the plurality of semi-products of microparticles further including a central layer formed by the third fluid, with the inner layer located between the outer layer and the central layer, and with each of the plurality of microparticle products including a shell layer formed by the second fluid and a core layer formed by the third fluid.

4. The method for producing microparticles as claimed in claim 3, with the nozzle including a tube assembly, with the tube assembly including a first tube and a second tube surrounded by the first tube, with a first fluid passageway defined between the first tube and the second tube, with a second fluid passageway defined in the second tube, with the first tube including a first end forming a first filling port intercommunicated with the first fluid passageway and a second end forming the plurality of first outlet ports intercommunicated with the first fluid passageway, with the second tube including a first end forming a second filling port and a second end forming a second outlet port, with a formation space defined between the second outlet port and the plurality of first outlet ports, with the third fluid forming a single-layer liquid film on the second outlet port, with the second fluid enveloping and shearing the single-layer liquid film formed on the second outlet port, thereby forming the plurality of dual-layer liquid films on the plurality of first outlet ports.

5. The method for producing microparticles as claimed in claim 4, with the second fluid flowing in the first fluid passageway toward the plurality of first outlet ports at a first speed, with the third fluid flowing through the second fluid passageway toward the second outlet port at a second speed, and with the first speed greater than the second speed.

6. The method for producing microparticles as claimed in claim 1, with the nozzle including a piezoelectric portion and an amplifying portion connected to the piezoelectric portion, wherein high frequency electric energy generated by a supersonic wave generator is transmitted to the piezoelectric portion and is converted by the piezoelectric portion into vibrational energy, and wherein the amplifying portion makes the plurality of liquid films on the plurality of first outlet ports absorb the vibrational energy.

7. The method for producing microparticles as claimed in claim 6, with the nozzle including a nozzle body having a first end and a second end opposite to the first end, and with the second fluid flowing from the first end toward the second end of the nozzle body and forming the plurality of liquid films on the plurality of first outlet ports.

8. The method for producing microparticles as claimed in claim 1, wherein the second fluid is a biodegradable polymer mixed with an active pharmaceutical ingredient in a particle or powder form.

9. The method for producing microparticles as claimed in claim 3, wherein the second fluid is a biodegradable polymer, and the third fluid is a fluid mixed with an active pharmaceutical ingredient in a liquid form.
